(19) United States
(12) Patent Application Publication
(10) Pub. No.: US 2023/0141054 A1
(43) Pub. Date: May 11, 2023

(54) METHOD FOR ADJUSTING A FILL LEVEL OF A LIQUID BALLOON OF A MEDICAL DEVICE, AND MEDICAL DEVICE COMPRISING A LIQUID BALLOON FOR FORMING ARTIFICIAL SPHINCTERS

(71) Applicant: A.M.I. Agency for Medical Innovations GmbH, Feldkirch (AT)

(72) Inventors: Martin HOHLRIEDER, Götzis (AT); Roland HUBER, Mäder (AT)

(73) Assignee: A.M.I. Agency for Medical Innovations GmbH, Feldkirch (AT)

(21) Appl. No.: 17/918,406

(22) PCT Filed: Apr. 13, 2021
(86) PCT No.: PCT/EP2021/059479

§ 371 Date: Oct. 12, 2022

(30) Foreign Application Priority Data
Apr. 23, 2020 (AT) A 96/2020

(51) Int. Cl. A61F 2/00

(52) U.S. Cl. CPC A61F 2/004; A61F 2250/0002; A61F 2250/0013

(57) ABSTRACT

A method for setting a fill level of a liquid balloon (1) is provided, in which the volume of a liquid-filled receiving space (10) of a pump unit (5) is changed by moving an actuating part (11) of the pump unit (5) by an electric motor (6) which is fed by a battery (7). An actuating process of the actuating part (11) is carried out by a control unit (8) which controls the electric motor (6). The actuating process includes travel control of the actuating part (11) for approaching a target position of the actuating part (11) and after this travel control a check as to whether the pressure of the liquid in the receiving space (10) lies within a permissible filling pressure range around the second filling pressure. If necessary, the position of the actuating part (11) is adjusted.

### INCORPORATION BY REFERENCE

The following documents are incorporated herein by reference as if fully set forth: PCT application No. PCT/EP2021/059479, filed Apr. 13, 2021; and Austrian Patent Application No. A 96/2020, filed Apr. 23, 2020.

### TECHNICAL FIELD

The invention relates to a method for setting a first fill level of a liquid balloon with a first filling pressure and a differently filled second fill level of the liquid balloon with a second filling pressure, wherein, for pumping liquid into the liquid balloon and for pumping liquid out of the liquid balloon, the volume of a liquid-filled receiving space of a pump unit, which receiving space is connected to the liquid balloon via a hose, is changed by moving an actuating part of the pump unit by means of an electric motor which is fed by a battery and, for setting the second fill level of the liquid balloon starting from the first fill level of the liquid balloon by a control unit which controls the electric motor, an actuating process of the actuating part is carried out, wherein the pressure of the liquid in the receiving space is detected. Furthermore, the invention relates to device comprising a liquid balloon, a pump unit which is connected to the liquid balloon via a hose for pumping liquid into the liquid balloon and for pumping liquid out of the liquid balloon, which pump unit has a receiving space which can be filled with liquid and the volume of which can be changed by moving an actuating part of the pump unit by means of an electric motor which is fed by a battery, an electronic control unit for controlling the electric motor and at least one pressure sensor for detecting the pressure of the liquid in the receiving space.

### BACKGROUND

In the medical sector, liquid balloons which can be filled with a liquid and can be closed to form a ring are used to form artificial sphincters, amongst other things for the urethra. In order to close the artificial sphincter, liquid is pumped into the inner chamber of the liquid balloon which is closed to form a ring around a body passage to be blocked in order to expand the inner wall of the liquid balloon against the hollow organ to be blocked. In order to open the body passage, the liquid is emptied from the chamber of the liquid balloon.

A manually operable pump for pumping the liquid out of the chamber of the liquid balloon is commonly implanted in the scrotum in an artificial urethral sphincter for male patients. The liquid can be pumped by pressing on a flexible part of the pump. This is usually followed by automatic closure of the urethra by way of liquid being pumped back into the inner chamber of the liquid balloon by a spring-elastic element of the pump.

Medical devices for narrowing or blocking off a body passage are also used elsewhere in the human body, for example for forming an artificial sphincter for an anus, possibly an artificial anus, as gastric bands for narrowing the gastrointestinal tract or as bands for closing a duct for bile. Medical devices of this kind are also referred to as cuffs, sleeves or artificial sphincters.

For example, U.S. Pat. No. 5,478,305 A discloses a medical device which is referred to as a cuff in said document and can be used to treat urinary or fecal incontinence. The cuff is produced from silicone. By filling the cuff with fluid, the pressure in the cavity of the cuff increases and closes the body passage. Examples of gastric bands can be found in EP 1 389 453 B1.

A method and a device of the kind mentioned at the outset can be found in WO 2017/205883 A1. A pump unit which is driven by an electric motor, a battery for power supply and an electronic control unit are arranged in a housing that can be implanted in the human body. The pressure of the liquid in the receiving space of the pump unit is detected by a pressure sensor. In order to fill a liquid balloon placed annularly around a body passage and thereby block the body passage, liquid is pumped into the chamber of the liquid balloon by means of the pump unit. Here, the pressure is set to a specific target value by the control unit.

### SUMMARY

The object of the invention is to provide a method and a device of the kind mentioned at the outset which render possible the lowest possible energy consumption of the battery. According to the invention, this is achieved by a method having one or more of the features described herein and by a device having one or more of the features described herein.

In the method according to the invention, the actuating process, which is executed by the control unit, comprises travel control of the actuating part of the pump unit in order to approach a target position of the actuating part. Here, a respective position (=actual position) of the actuating part is detected by means of a travel sensor. Such travel control in order to approach a target position of the actuating part can be carried out in a very efficient and energy-saving manner. In particular, the control can be carried out as discontinuous control. In order to achieve a second fill level starting from a first fill level of the liquid balloon, it is particularly preferred to carry out two-point control, i.e. the control unit controls the electric motor only using the switching states “on” and “off”. The control variable here is the position of the actuating part, which position is detected by means of the travel sensor.

Following this travel control, a check is made as to whether the pressure of the liquid in the receiving space lies within a permissible filling pressure range. The filling pressure range here extends around the second filling pressure to which the pressure of the liquid is intended to be brought in the second filling state of the liquid balloon. If the pressure in the receiving space of the pump unit does not lie within this permissible filling pressure range, the position of the actuating part is adjusted. This process (checking the pressure in the receiving space and, if necessary, adjusting the position of the actuating part) can be carried out repeatedly.

In order to determine the target position of the actuating part when, starting from a first fill level of the liquid balloon with a first filling pressure, a differently filled second fill level of the liquid balloon with a second filling pressure is intended to be achieved, a characteristic curve which is stored in the control unit and shows the relationship between the position of the actuating part and the pressure of the liquid in the receiving space of the pump unit is advantageously used. This characteristic curve, which is preferably set as a straight line of which the gradient is stored in the control unit, is expediently recorded in a preceding learning cycle. In a learning cycle of this kind, two or more positions of the actuating part can be approached and the pressure of the liquid in the receiving space can be determined in each case. A straight line can be determined from these recorded pairs of values, there being extremely small deviations from these pairs of values in relation to said straight line (for example, the sum of the magnitudes of the deviations or the sum of the squares of the deviations can be minimized).

An advantageous development provides that, if the position of the actuating part is adjusted during the course of an actuating process, the gradient of the straight line is changed in proportion to the magnitude of the adjustment of the position of the actuating part. The characteristic curve is therefore adapted for subsequent actuating processes.

If the position of the actuating part needs to be adjusted after the travel control of the actuating part has been carried out because the pressure in the receiving space lies outside the permissible filling pressure range, an advantageous embodiment of the invention provides that the target position of the actuating part is modified and further travel control of the actuating part for approaching the modified target position is carried out by the control unit.

In an advantageous device according to the invention, it is provided that the pump device comprises a bellows within which the receiving space for the liquid is situated, wherein an end piece of the bellows is rigidly connected to the actuating part. Here, the actuating part can have, in particular, an external thread which interacts with an internal thread of an actuating ring which can be rotated by the electric motor. Here, the actuating ring can have a toothing, in particular on its outer circumference, which interacts with a worm gear which is driven by the electric motor. Robust and precise adjustment of the actuating part can be achieved in this way, with the position of the actuating part being fixed in the state in which the electric motor is not supplied with power.

A device according to the invention can advantageously form an artificial sphincter, in particular a urethral sphincter, wherein the liquid balloon is in the form of a flexible band which can be closed to form a ring and has a longitudinally running inner chamber which can be filled with the liquid.

Due to the low level of power consumption made possible by the invention, a long service life can be achieved before the battery needs to be replaced. If the battery is designed to be rechargeable, a less frequent need for charging the battery can be achieved.

### BRIEF DESCRIPTION OF THE DRAWINGS

Further advantages and details of the invention are explained below with reference to the attached drawing, in which:

FIG. 1 shows a device according to the invention in the implanted state;

FIGS. 2 and 3 show a side view and view of the motorized operating unit;

FIG. 4 shows a section along the line AA from FIG. 3 in a first position of the actuating part;

FIG. 5 shows a section corresponding to FIG. 4 in a second position of the actuating part;

FIG. 6 shows an exploded illustration;

FIG. 7 shows an oblique view of the plate of the travel sensor which is electrically connected to the electronic control unit and has the spiral conductor track;

FIG. 8 shows an oblique view of the bellows of the pump unit;

FIG. 9 shows an oblique view of the liquid balloon which is closed to form a ring;

FIG. 10 shows an axial view of the liquid balloon which is closed to form a ring in the unfilled state of the inner chamber;

FIG. 11 shows a section along the line BB from FIG. 10;

FIG. 12 shows an illustration corresponding to FIG. 10 in the filled state of the inner chamber;

FIG. 13 shows a side view of the manual operating unit;

FIG. 14 shows a plan view of the manual operating unit;

FIG. 15 shows a section along the line CC from FIG. 13 in the non-operated state of the manual operating unit;

FIG. 16 shows a section analogous to FIG. 15 in the operated state of the manual operating unit;

FIG. 17 shows a section along the line DD from FIG. 14 in the closed state of the shut-off valve;

FIG. 18 shows a section corresponding to FIG. 17 in the open state of the shut-off valve;

FIG. 19 shows an exploded illustration of the manual operating unit;

FIG. 20 shows a graph for illustrating the learning cycle and the determination of the characteristic curve; and

FIG. 21 shows a graph for explaining the setting of a second fill level starting from a first fill level of the liquid balloon.

### DETAILED DESCRIPTION

An exemplary embodiment of a device according to the invention is illustrated in the figures.

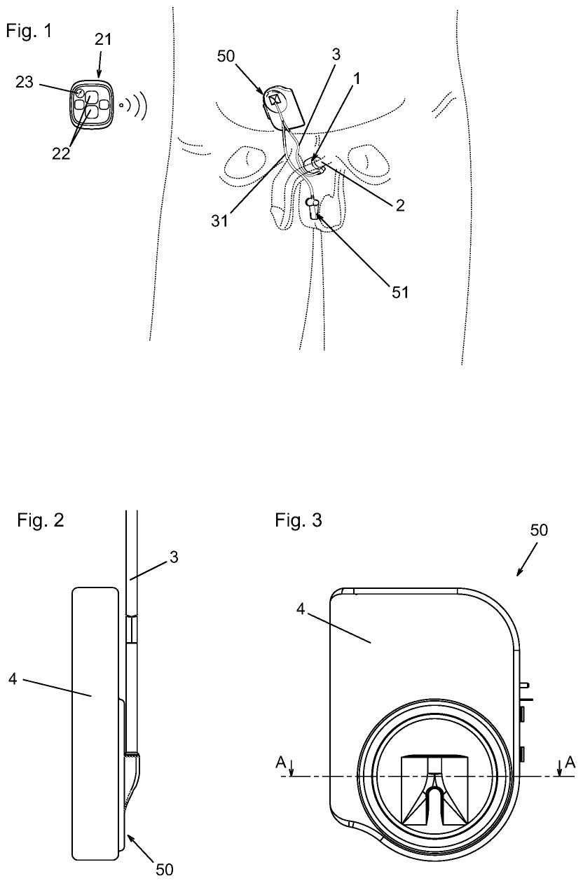

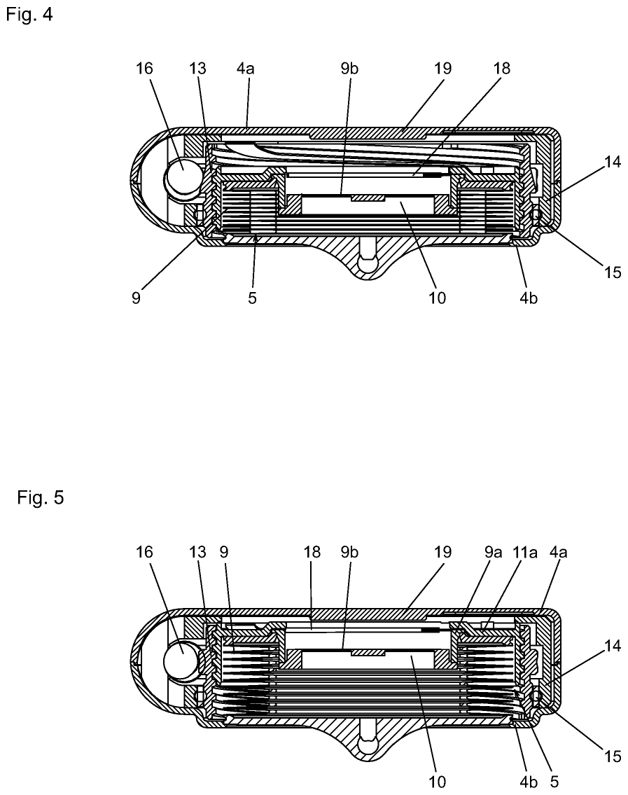

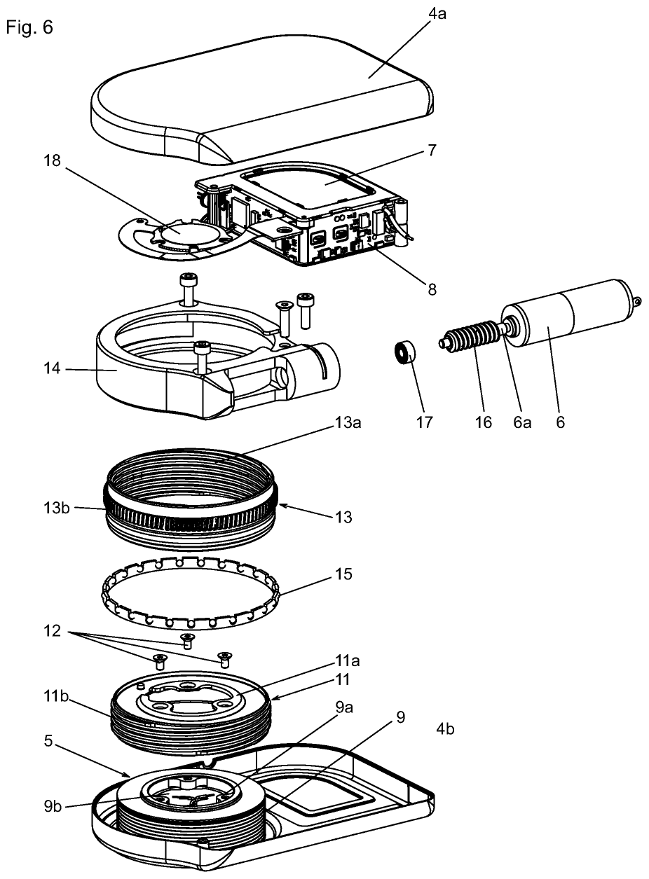

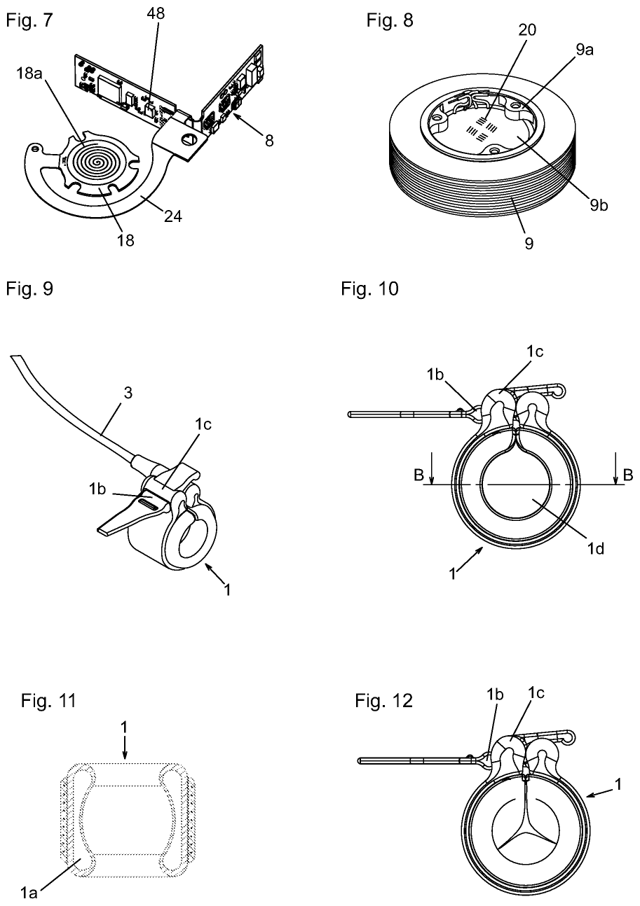

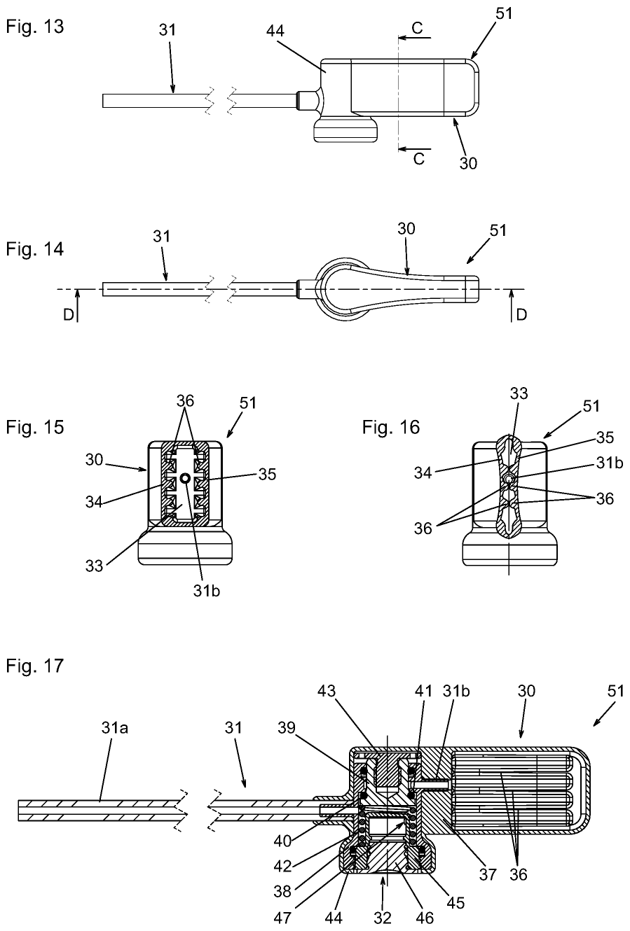

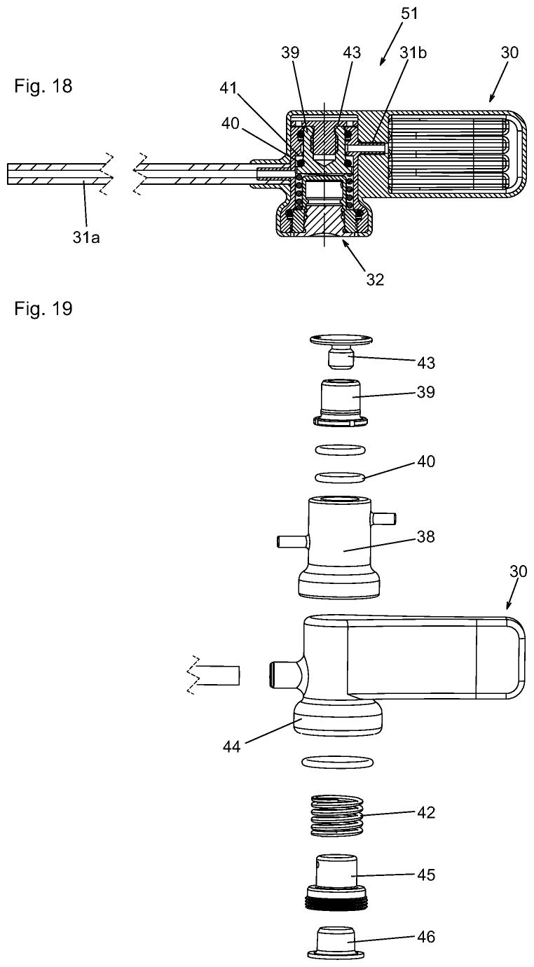

A liquid balloon 1 is designed in the form of a flexible band having a longitudinally running inner chamber 1a. The liquid balloon can be closed to form a ring by means of closure parts 1b, 1c which are arranged at the two end regions, wherein said liquid balloon can be placed in a ring shape around a body passage, here the urethra 2, cf. FIG. 1. When the liquid balloon 1 is closed to form a ring and the chamber 1a is emptied, the liquid balloon 1 which is closed to form a ring has a passage opening 1d, cf. FIG. 10. The size of the passage opening 1d can be reduced by pumping liquid into the chamber 1a, cf. FIG. 11.

A pump unit 5, which is connected to the liquid balloon 1 via a hose 3 and is arranged in a motorized operating unit 50 which can be implanted into the body, is used for pumping liquid into the liquid balloon 1 and for pumping liquid out of the liquid balloon 1. This motorized operating unit 50 has a housing 4 in which, apart from the pump unit 5, an electric motor 6 for driving the pump unit 5, a battery 7 for supplying power to the electric motor 6 and an electronic control unit 8, which controls the electric motor 6 and therefore also the pump unit 5 and is likewise fed by the battery 7, are arranged.

In the exemplary embodiment, the housing 4 comprises a half-shell-shaped upper housing part 4a and a half-shell-shaped lower housing part 4b which are connected to one another in a gas-tight manner. The interior of the housing 4 is therefore insulated from the area surrounding the housing 4 in a gas-tight manner.

The pump unit 5 has a bellows 9, in particular folding bellows. The bellows 9 encloses a receiving space 10 in which a liquid, e.g. water, is located. Furthermore, the pump unit 5 has an actuating part 11 which is rigidly connected to an end piece 9a of the bellows, in the exemplary embodiment by means of screws 12. The actuating part 11 has a bottom 11a which is connected to the end piece 9a of the bellows 9 and has a sleeve section which surrounds the outside of the bellows 9 and has an external thread lib. An internal thread 13a of an actuating ring 13 is in engagement with the external thread 11b. The actuating ring 13 is rotatably mounted in a bearing housing 14. A ball bearing 15 can be provided for rotatably mounting the actuating ring 13 in the bearing housing 14, in particular as illustrated. The actuating ring 13 also has a toothing 13b which surrounds the outside of said actuating ring. A worm gear 16 which is driven by the electric motor 6 is in engagement with the toothing 13b of the actuating ring. The worm gear 16 can be arranged directly on the motor shaft 6a of the electric motor 6.

In the exemplary embodiment, the electric motor 6 is held on the bearing housing 14 and the motor shaft 6a is mounted at the end side by a bearing ring 17 such that it can rotate with respect to the bearing housing 14.

Strain gauges are fitted, for example sputtered, onto the end membrane 9b of the bellows 9, which end membrane delimits the interior of the bellows 9 in the axial direction of the bellows 9, as indicated in FIG. 8. These strain gauges provide a pressure sensor 20 for detecting the pressure which acts between the receiving space 10 of the bellows 9 and the space surrounding the bellows 9 (=interior of the housing 4). Pressure sensors of this kind which are formed by means of strain gauges are known per se.

The pressure sensor 20 is electrically connected to the control unit 8.

A plate 18 which has a spiral conductor track 18a is rigidly connected to the actuating part 11. In the exemplary embodiment, the plate 18 is arranged between the actuating part 11 and the end piece 9a of the bellows 9. The plate 18 is used as a sensor for the distance from the section 19 of the wall of the housing 4, which section is situated opposite the plate. Therefore, an inductive travel sensor for detecting the position of the actuating part 11 is formed by the plate 18 in connection with the section 19 of the wall of the housing 4.

The conductor track 18a of the plate 18 is electrically connected to the control unit 8. In the exemplary embodiment, a flexible conductor track carrier 24 which runs from the plate 18 in the form of a helicoidal surface to the control unit 8 is provided for this purpose.

When the electric motor 6 is operated by the control unit 8, this electric motor adjusts the actuating part 11 in the axial direction of the helical gear mechanism and the bellows 9 via the worm gear mechanism, which is formed between the electric motor and the actuating ring 13, and the helical gear mechanism, which is formed between the actuating ring 13 and the actuating part 11. As a result, the volume of the receiving space 10 is changed.

A remote control arrangement 21 is preferably provided for operating the device. Wireless data transmission takes place between the remote control arrangement 21 and the control unit 8. The remote control arrangement 21 has operator control elements 22. These operator control elements can be used to switch over between an open state and a closed state of the device. A first fill level of the liquid balloon 1 with a first filling pressure prevails in the open state and a differently filled second fill level of the liquid balloon with a second filling pressure which is higher than at the first fill level prevails in the closed state.

An air pressure sensor 23 for detecting the atmospheric pressure pU is also arranged in the remote control arrangement 21, the importance of which will be explained in more detail further below.

In order to detect the air pressure pG in the hermetically sealed housing 4, a housing air pressure sensor 48 is arranged in the housing 4, the importance of which will be explained in more detail further below.

In addition to the motorized operating unit 50, a manual operating unit 51 is also preferably provided, with which the device can be manually switched between the open state and the closed state, particularly in the event that the motorized operating unit 50 should fail. The manual operating unit 51 is explained in more detail further below.

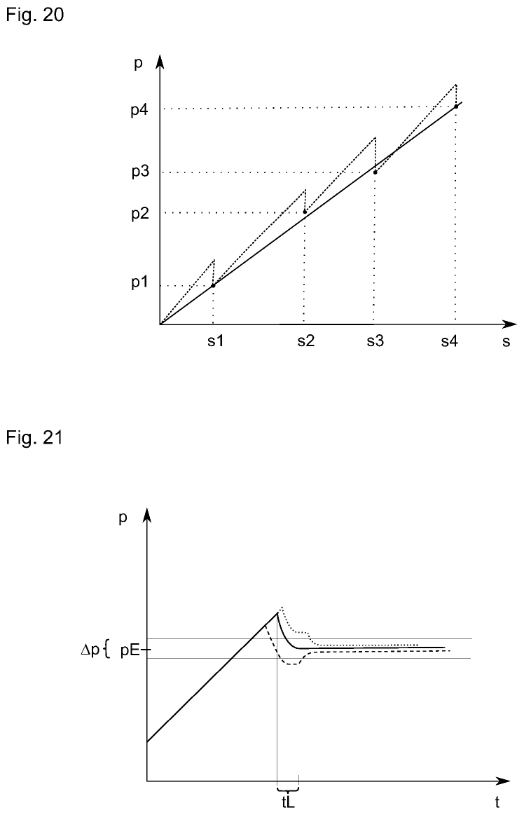

A learning cycle for determining a characteristic curve, which shows the relationship between the position of the actuating part and the pressure of the liquid in the receiving space of the pump device, is explained below with reference to FIG. 20. A learning cycle of this kind is carried out in particular when the device is put into operation for the first time and can optionally be repeated at later times. In order to carry out the learning cycle, the device is filled with liquid. The learning cycle is preferably carried out in the implanted state of the device. In principle, it would also be conceivable and possible to carry out the learning cycle with the device located outside the body. For this purpose, the liquid balloon could be placed around an, in particular tubular, element which corresponds to the urethra in terms of its dimensions and possibly its elasticity.

In order to record the characteristic curve, several different positions of the actuating part are approached. By way of detecting the actual positions of the actuating part 11 by means of the travel sensor 18, 19, travel control for approaching the desired target positions s1, s2, s3 and s4 is carried out by the control unit 8, specifically starting from an initial position in which the lowest filling pressure prevails in the liquid balloon. Starting from this initial position, the travel control is carried out by the control unit 8 as discontinuous control, preferably as two-point control which uses only “motor on” and “motor off” as control values. When a respective target position s1, s2, s3 and s4 is reached, the process waits for a certain relaxation time. Within this relaxation time, the liquid pressure in the system equalizes, so that there is a drop in the pressure in the receiving space of the pump unit. The pressure p1, p2, p3, p4 at the respective position s1, s2, s3 and s4 approached is then recorded and the position and pressure values are stored.

The characteristic curve is set as a straight line. An optimized straight line is therefore determined from the stored pairs of values, in which optimized straight line the sum of the magnitudes of the deviations of the pressure values p1, p2, p3, p4 or the sum of the squares of the deviations of the pressure values p1, p2, p3, p4 from the straight line is the smallest. The gradient k of this straight line is stored.

In principle, it would also be conceivable and possible to record the characteristic curve by approaching just two positions of the actuating part and determining the associated pressure values, wherein the straight line runs through these two pairs of values. However, it is preferred to record the characteristic curve by approaching more than two positions of the actuating part.

After the characteristic curve has been recorded, it is also possible to change the quantity of liquid in the device in order to adapt the working range of the pump unit 5 (that is to say the range over which the actuating part 11 is moved). The gradient of the straight line of the characteristic curve can be assumed to be unchanged here. However, renewed recording of the characteristic curve is likewise conceivable and possible.

The setting of a second fill level of the liquid balloon 1 starting from a first fill level is explained below with reference to FIG. 21. FIG. 21 shows the course of the pressure p of the liquid in the receiving space 10 of the pump unit 5 as a function of time t.

At the first fill level of the liquid balloon 1, the actuating part is in an initial position sA. The pressure p of the liquid in the receiving space 10 of the pump unit 5 has an initial value pA. In the static state, the pressure pA of the liquid in the receiving space corresponds to the filling pressure in the liquid balloon.

In order to now set the second fill level of the liquid balloon 1, the end position sE to which the actuating part 11 is intended to be moved is determined on the basis of the gradient of the characteristic curve. The end position sE is calculated as follows:

sE=sA+k*(pE−pA).

pE corresponds to the desired second filling pressure here.

In particular, it can be provided that the liquid balloon 1 is intended to assume only two different states which correspond to an open state and a closed state of the device, wherein the filling pressure pE in the liquid balloon in the closed state of the device is greater than the filling pressure pA in the open state.

Travel control of the actuating part 11 is now carried out by the control unit 8 with the desired end position sE as the target position. This travel control is advantageously carried out as discontinuous control, preferably as two-point control with the control values “motor on” and “motor off”.

With this travel control, the actual position of the control part 11 is continuously detected by means of the travel sensor 18, 19 and initially the control value “motor on” is sent to the electric motor 6. The end position sE of the actuating part 11 minus a specified latency distance is set as the switch-off position. When the actual position of the actuating part 11 reaches this switch-off position, the electric motor 6 is switched off. The travel control can then be terminated. However, after the electric motor 6 has been switched off, a check can also be made as to whether the desired target position sE has been reached within a specified tolerance range and, if not, the latency distance for subsequent travel control operations can be adapted and/or further travel control for reaching the target position within the tolerance range can be carried out.

After completion of the travel control, the process waits for a specified latency time tL in which the liquid pressure in the device can be equalized. During the latency time tL, the pressure of the liquid in the receiving space will therefore drop, cf. FIG. 21. After this latency time has elapsed, the pressure is detected by means of the pressure sensor 20. The solid line in FIG. 21 shows the situation in which the pressure p in the receiving space after the latency time tL lies within a permissible filling pressure range Δp around the desired filling pressure pE. It is then not necessary to adjust the position of the actuating part.

The desired filling pressure pE preferably lies in the middle of the filling pressure range Δp. For example, the filling pressure range Δp can be in the range of +/−1% to +/−5% around the value of the filling pressure pE.

The situations in which the pressure p in the receiving space lies above or below the permissible filling pressure range Δp around the desired pressure pE after the latency time has elapsed are indicated with dotted or dashed lines in FIG. 21. In these situations, the position of the actuating part 11 is adjusted, so that after the position of the actuating part 11 has been adjusted, the pressure p in the receiving space lies within the permissible filling pressure range.

This adjustment of the position of the actuating part is preferably performed by further travel control of the actuating part, which in turn is advantageously carried out as discontinuous control, preferably as two-point control. However, it would also be conceivable and possible for the position of the actuating part to be adjusted in specified steps, that is to say for the actuating part 11 to be moved by a specified step in the appropriate direction until it is established that the pressure p in the receiving space lies within the filling pressure range Δp.

If the position of the actuating part is adjusted, the gradient k of the characteristic curve is preferably adapted. The new gradient kb can be determined from the previous gradient ka using the following relationship:

kb=a*ka+(1−a)*(sE−sA)/(pE−pA).

The constant a lies between 0.1 and 0.9, preferably between 0.5 and 0.9, here.

This can also compensate for osmosis effects, owing to which the volume of the liquid in the device could possibly be changed somewhat.

In order to determine the pressure of the liquid in the receiving space 10 of the pump unit 5, the air pressure in the housing 4 and a change in the atmospheric pressure are taken into account. The pressure sensor 20 detects the differential pressure between the two sides of the end membrane 9b of the bellows 9. Since the atmospheric pressure pU acts on the liquid balloon 1 and therefore also on the liquid contained in the liquid balloon, the pressure acting on the end membrane 9b on the liquid side is the sum of the liquid pressure p and the atmospheric pressure pU. The air pressure pG prevailing within the housing 4 is applied to the opposite side of the end membrane 9b. Since the housing 4 is hermetically sealed, this air pressure changes only as a function of the volume which the bellows 9 occupies in its respective position, that is to say in the respective position of the actuating part 11. The pressure value pS output by the pressure sensor 20 is therefore:

pS=p+pU−pG.

In order to determine the pressure of the liquid in the receiving space 10 of the pump units 5, the atmospheric pressure pU which is output by the air pressure sensor 23 is therefore subtracted from the pressure value pS which the pressure sensor 20 outputs, and the air pressure pG in the interior of the housing (and outside the bellows) which is output by the housing air pressure sensor 48 is added.

The pressure p of the liquid prevailing in the receiving space 10 of the pump unit 5 is preferably stored after each actuating process which is carried out by the control unit 8. Before a renewed actuating process, a check is made as to whether the current pressure p of the liquid in the receiving space 10 of the pump unit 5 is still within a tolerance range around the stored value of the pressure p. If this is not the case, the reasons for this may essentially be as follows:

The liquid balloon 1 could have been opened by means of the manual operating unit 51. If the liquid is then pumped back into the liquid balloon 1 by means of the manual operating unit 51, the pressure p of the liquid in the receiving space 10 can be checked once again. If it is not possible to achieve the situation that the pressure p lies in the specified range in this way, it is assumed that there is a leak and therefore the device needs to be checked.

The manual operating unit 51 has a flexible pump body 30 which is connected to the liquid balloon 1 via a connecting line 31. Furthermore, the manual operating unit 51 has a manually openable shut-off valve 32 which is arranged between two sections 31a, 31b of the connecting line 31. In an open position of the shut-off valve 32, the two sections 31a, 31b of the connecting line are connected to one another in a liquid-conducting manner. In a closed position of the shut-off valve 32, the liquid-conducting connection between the two sections 31a, 31b is interrupted.

The first section 31a of the connecting line 31, which first section is formed by a hose, connects the shut-off valve 32 to the inner chamber 1a of the liquid balloon 1. The second section 31b of the connecting line 31, which second section is substantially shorter than the first section 31a, connects the shut-off valve 32 to a pump chamber 33 which is arranged in the pump body 30.

The volume of the pump chamber 33 which is arranged in the pump body 30 can be reduced by an operating force which can be manually exerted by the user and acts on the pump body 30. For this purpose, in particular, two opposite side walls 34, 35 of the pump body 30 can be pressed together until they are brought into contact. At least one of the side walls 34, 35, preferably both side walls 34, 35, has/have a plurality of raised portions 36 on their side situated in the interior of the pump chamber 33. When the side walls 34, 35 are in a state in which they are fully pressed together, they rest against one another via the raised portions 36. The raised portions may be, in particular, fins which are spaced apart from one another and preferably run in parallel.

The second section 31b of the connecting line 31, which second section runs through a connecting wall 37 of the pump body, which connecting wall connects the opposite side walls 34, 35, opens into the pump chamber 33 at a point which, in the state in which the opposite side walls 34, 35 are fully pressed together, lies in the region of a recess which is located between or next to the raised portions 36 of the at least one side wall 34, 35. In a sectional view orthogonal to the direction of longitudinal extent of the pump body 30, the connecting line 31 opens into the pump chamber 33 in the region between two raised portions 36 of the same side wall 34, 35. As a result, when the side walls 34, 35 are pressed together until the side walls 34, 35 come into contact with one another, liquid can flow out of the pump chamber 33 (with the shut-off valve 32 open).

It would also be conceivable and possible for the raised portions 36 to end at a distance from the connecting wall 37.

The shut-off valve 32 has a closure member 39 which is displaceably mounted in a valve housing 38. Starting from a closed position, in which the closure member 39 is sealed off from a valve seat 41 by means of a seat seal 40, the closure member 39 can be displaced against the force of a return spring 42 into an open position. For this purpose, an operating button 43 which is connected to the closure member 39 or formed in one piece with it is pressed in.

The return spring 42 is supported on a closure part 45 which is screwed into the valve housing 38. The closure part is of pot-shaped design and sealed off from the surrounding area by a port 46. The port 46 is used to introduce liquid into the device by piercing it with a cannula. Openings 47 are made in the pot-shaped closure part 45 here.

The shut-off valve 32 and the pump body 30 are advantageously formed integrally with one another. This integral unit also comprises the second section 31b of the connecting line 31.

A sheath 44 which sheaths the shut-off valve 32 and is formed in one piece with the pump body 30 is preferably present.

In order to manually empty the liquid balloon 1, the shut-off valve 32 is opened by pressing the operating button 43, as a result of which liquid can flow out of the chamber 1a of the liquid balloon into the pump chamber 33 of the pump body 30.

In order to fill liquid into the chamber 1a of the liquid balloon 1, the shut-off valve 32 is opened by pressing the operating button 43 and the side walls 34, 35 of the pump body 30 are pressed together until they make contact with one another. After the operating button 43 is released, liquid is blocked from flowing back out of the chamber 1a of the liquid balloon 1.

It would also be conceivable and possible for the shut-off valve 32 to be of self-opening design when the pump body 30 is compressed, for example by way of appropriately dimensioning the return spring 42.

When designed as an artificial urethral sphincter, the manual operating unit 51 is preferably implanted in the scrotum.

The manual operating unit 51 together with the connecting line 31 could also be dispensed with in principle.

### LEGEND FOR THE REFERENCE NUMERALS

1 Liquid balloon
1a Chamber
1b Closure part
1c Closure part
1d Passage opening
2 Urethra
3 Hose
4 Housing
4a Upper housing part
4b Lower housing part
5 Pump unit
6 Electric motor
6a Motor shaft
7 Battery
8 Control unit
9 Bellows
9a End piece
9b End membrane
10 Receiving space
11 Actuating part
11a Bottom
11b External thread
12 Screw
13 Actuating ring
13a Internal thread
13b Toothing
14 Bearing housing
15 Ball bearing
16 Worm gear
17 Bearing ring
18 Plate
18a Conductor track
19 Section
20 Pressure sensor
21 Remote control arrangement
22 Operator control element
23 Air pressure sensor
24 Conductor track carrier
30 Pump body
31 Connecting line
31a First section
31b Second section
32 Shut-off valve
33 Pump chamber
34 Side wall
35 Side wall
36 Raised portion
37 Connecting wall
38 Valve housing
39 Closure member
40 Seat seal
41 Valve seat
42 Return spring
43 Operating button
44 Sheath
45 Closure part
46 Port
47 Opening
48 Housing air pressure sensor
50 Motorized operating unit
51 Manual operating unit

1. A method for setting a first fill level of a liquid balloon with a first filling pressure and a differently filled second fill level of the liquid balloon with a second filling pressure, the method comprising:
for pumping liquid into the liquid balloon and for pumping liquid out of the liquid balloon, changing a volume of a liquid-filled receiving space of a pump unit which is connected to the liquid balloon via a hose by moving an actuating part of the pump unit using an electric motor which is powered by a battery;
for setting the second fill level of the liquid balloon starting from the first fill level of the liquid balloon by a control unit which controls the electric motor, carrying out an actuating process of the actuating part including detecting a pressure of the liquid in the receiving space; and
the actuating process comprising travel control of the actuating part for approaching a target position of the actuating part, including detecting a respective position of the actuating part using a travel sensor, and after the travel control, checking whether the pressure of the liquid in the receiving space lies within a permissible filling pressure range around the second filling pressure, and in the event that the pressure of the liquid in the receiving space lies outside the permissible filling pressure range around the second filling pressure, adjusting the position of the actuating part.

2. The method as claimed in claim 1, further comprising using a characteristic curve which is stored in the control unit that shows a relationship between the position of the actuating part and the pressure of the liquid in the receiving space for determining the target position of the actuating part.

3. The method as claimed in claim 2, wherein the characteristic curve is set as a straight line of which a gradient (k) is stored by the control unit.

4. The method as claimed in claim 3, wherein if the position of the actuating part is adjusted during the actuating process, the gradient (k) of the straight line is changed in proportion to a magnitude of the adjustment of the position of the actuating part.

5. The method as claimed in claim 1, wherein, for adjusting the position of the actuating part, if the pressure in the receiving space lies outside the filling pressure range around the second filling pressure, modifying the target position of the actuating part carrying out further travel control of the actuating part for approaching the modified target position using the control unit.

6. The method as claimed in claim 1, wherein the travel control for setting the second fill level starting from the first fill level is carried out as discontinuous control.

7. The method as claimed in claim 1, further comprising storing the pressure of the liquid in the receiving space in the control unit after completion of an actuating process and comparing the stored pressure with the pressure currently prevailing in the receiving space before carrying out a further actuating process.

8. A device comprising:
a liquid balloon;
a pump unit connected to the liquid balloon via a hose for pumping liquid into the liquid balloon and for pumping liquid out of the liquid balloon, the pump unit has a receiving space is fillable with liquid and a volume of which is changeable by moving an actuating part of the pump unit via an electric motor powered by a battery;
an electronic control unit configured to control the electric motors;
at least one pressure sensor configured to detect a pressure of the liquid in the receiving space; and
a travel sensor configured to detect a position of the actuating part.

9. The device as claimed in claim 8, wherein the pump unit has a bellows within which the receiving space for the liquid is situated, and an end piece of the bellows is rigidly connected to the actuating part.

10. The device as claimed in claim 9, wherein the pressure sensor has at least one strain gauge attached to an end membrane which delimits an interior of the bellows in an axial direction of the bellows.

11. The device as claimed in claim 10, further comprising an air pressure sensor configured to detect atmospheric pressure and a housing air pressure sensor configured to detect an air pressure in a gas-tight-insulated interior of a housing in which the pump unit is arranged, and, for determining the pressure of the liquid in the receiving space of the pump unit, the electronic control unit is configured to subtract the atmospheric pressure which is detected by the air pressure sensor from the measured value which is output by the pressure sensor, and to add the air pressure in the housing which is detected by the housing air pressure sensor.

12. The device as claimed in claim 9, wherein the actuating part has an external thread which interacts with an internal thread of an actuating ring which is rotatable by the electric motor.

13. The device as claimed in claim 9, wherein the travel sensor has a plate which is rigidly connected to the actuating part and has a spiral conductor track which is situated opposite a static, electrically conductive counter-plate.

14. The device as claimed in claim 9, wherein the device forms an artificial sphincter, the liquid balloon comprises a flexible band which is closeable to form a ring and has a longitudinally running inner chamber which forms the liquid balloon and is fillable with the liquid.

15. The device as claimed in claim 9, wherein the control unit is configured to execute an actuating process of the actuating part via the electric motor in order to, starting from a first fill level of the liquid balloon with a first filling pressure, set a second fill level of the liquid balloon with a second filling pressure, and the actuating process comprises travel control of the actuating part for approaching a target position of the actuating part, after said travel control a check as to whether the pressure of the liquid in the receiving space lies within a permissible filling pressure range around the second filling pressure and, if the pressure of the liquid in the receiving space lies outside the permissible filling pressure range around the second filling pressure, adjusts the position of the actuating part.
